(19) United States
(12) Patent Application Publication
(10) Pub. No.: US 2023/0132050 A1
(43) Pub. Date: Apr. 27, 2023

(54) RADIO FREQUENCY-BASED THERAPEUTIC DEVICE FOR THE TREATMENT OF NEUROLOGICAL CONDITIONS

(71) Applicant: Remarkable Technologies, Inc., Avon, CT (US)

(72) Inventor: Eric A. Knight, Unionville, CT (US)

(21) Appl. No.: 17/973,742

(22) Filed: Oct. 26, 2022

Related U.S. Application Data
(60) Provisional application No. 63/271,958, filed on Oct. 26, 2021.

(51) Int. Cl. A61N 1/36; H01Q 1/27; H01Q 21/20

(52) U.S. Cl. CPC A61N 1/36082; H01Q 1/273; H01Q 21/20

(57) ABSTRACT

Methods, devices, and processor-readable storage media for a radio frequency-based (RF-based) therapeutic for the treatment of neurological conditions are provided herein. An example device includes one or more antenna elements sized and configured for one or more operational frequencies; at least one RF generator-controller unit; at least one switching element coupled with at least a portion of the one or more antenna elements; and a distributed functional architecture and/or at least one cable, wherein the distributed functional architecture and/or the at least one cable facilitates transmission of one or more of RF emissions, one or more antenna control voltages, and one or more system-control signals between the at least one RF generator-controller unit and the at least one switching element in connection with at least a portion of the one or more antenna elements.

### CROSS-REFERENCE TO RELATED APPLICATIONS

The present application claims priority to U.S. Provisional Application Ser. No. 63/271,958, filed Oct. 26, 2021, entitled “Radio Frequency-Based Therapeutic Device for the Treatment of Neurological Conditions,” the entire contents of which are incorporated by reference herein for all purposes.

### FIELD

The field relates generally to healthcare technology, and more particularly to treatments of neurological conditions.

### BACKGROUND

Neurological conditions such as, for example, Alzheimer's diseases and other forms of dementia, affect an increasing number of individuals while conventional treatment options remain limited in number and efficacy.

### SUMMARY

Illustrative embodiments provide a radio frequency-based (RF-based) therapeutic device for the treatment of neurological conditions. An example device can include one or more antenna elements sized and configured for one or more operational frequencies; at least one RF generator-controller unit; at least one switching element coupled with at least a portion of the one or more antenna elements; and at least one cable, wherein the at least one cable facilitates transmission of one or more of RF emissions, one or more antenna control voltages, and one or more system-control signals between the at least one RF generator-controller unit and the at least one switching element in connection with at least a portion of the one or more antenna elements.

Another example device can include one or more antenna elements sized and configured for one or more operational frequencies; at least one RF generator-controller unit; at least one switching element coupled with at least a portion of the one or more antenna elements; and a distributed functional architecture, wherein the distributed functional architecture facilitates transmission of one or more of RF emissions, one or more antenna control voltages, and one or more system-control signals between the at least one RF generator-controller unit and the at least one switching element in connection with at least a portion of the one or more antenna elements.

An example method can include rendering at least a portion of one or more antenna elements and at least a portion of at least one switching element within a given proximity of a user; configuring at least one RF generator-controller unit to deliver at least one therapeutic regimen; and activating the at least one RF generator-controller unit based at least in part on the rendering of the at least a portion of the one or more antenna elements and the at least a portion of the at least one switching element within the given proximity of the user; wherein the method is carried out by at least one RF-based therapeutic device.

Illustrative embodiments can provide significant advantages relative to conventional neurological treatment options. For example, challenges associated with ineffective treatments are overcome through the use of a portable treatment device that can be used in hospital and non-hospital (e.g., residential) settings, implemented with configurations that enable the user-therapeutic section(s) of the device (or the entire device itself) to be cleaned and/or washed, and wherein such a device provides a non-pharmacological therapeutic approach that reduces and/or eliminates the need for medication consumption and complications related thereto.

These and other illustrative embodiments described herein include, without limitation, methods, apparatus, networks, systems, and processor-readable storage media.

### BRIEF DESCRIPTION OF THE DRAWINGS

FIG. 1A, FIG. 1B, and FIG. 1C are diagrams showing illustrative implementations of example embodiments;

FIG. 2 is a block diagram illustrating an example circuit of a treatment device in an example embodiment;

FIG. 3 is a block diagram illustrating an example circuit of a treatment device in an example embodiment;

FIG. 4 is a block diagram illustrating another example circuit of a treatment device in an example embodiment; and

FIG. 5 is a block diagram illustrating another example circuit of a treatment device in an example embodiment; and

FIG. 6 is a flow diagram of a process for implementing a treatment device in an illustrative embodiment of the invention.

### DETAILED DESCRIPTION

Example and/or illustrative embodiments will be described herein with reference to exemplary circuits and/or other types of electrical components and devices. It is to be appreciated, however, that the invention is not restricted to use with the particular illustrative device configurations shown.

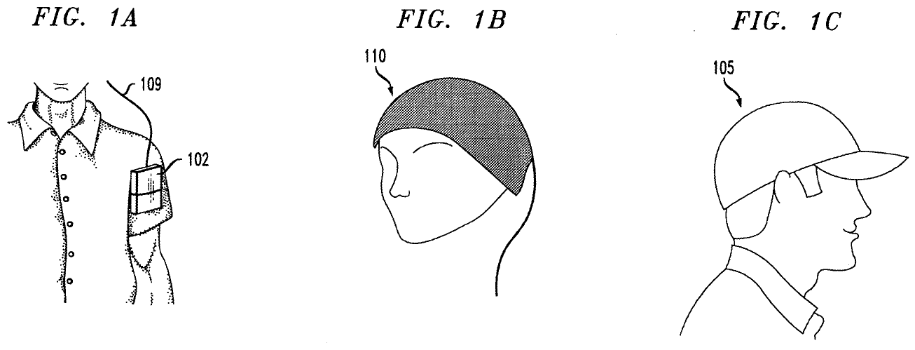

FIG. 1A, FIG. 1B, and FIG. 1C are diagrams showing illustrative implementations of example embodiments. As further detailed herein, in at least one embodiment, a treatment device (e.g., as depicted in the FIG. 1A, FIG. 1B, and/or FIG. 1C embodiments) can be implemented using at least one processing device. Each such processing device can include at least one processor and at least one associated memory, and can implement one or more functional software modules or components for controlling certain features of the treatment device.

In such an embodiment, the processor can include, for example, a microprocessor, a microcontroller, an application-specific integrated circuit, a field-programmable gate array or other type of processing circuitry, as well as portions or combinations of such circuitry elements. The memory can include, for example, random access memory (RAM), read-only memory (ROM) or other types of memory, in any combination. Additionally, the memory can also be viewed as examples of processor-readable storage media, which can store executable computer program code and/or other types of software programs.

Further, in one or more embodiments, each such processing device can also include a network interface, for example, for connection with one or more user devices or other types of devices. Also, it is to be appreciated that the term “user” herein is intended to be broadly construed so as to encompass, for example, human, hardware, software or firmware entities, as well as various combinations of such entities.

A network, such as detailed herein in connection with one or more embodiments, is assumed to include a portion of a global computer network such as the Internet, although other types of networks can be part of the network, including a wide area network (WAN), a local area network (LAN), a satellite network, a telephone or cable network, a cellular network, a wireless network such as a Wi-Fi or WiMAX network, or various portions or combinations of these and other types of networks. Additionally, as noted, a treatment device such as described herein can be coupled to one or more additional devices such as mobile telephones, laptop computers, tablet computers, desktop computers or other types of computing devices and/or user devices.

Further, in at least one embodiment, the treatment device can have an associated database configured to store data related to treatment sessions and/or users. In such an embodiment, the database can be implemented using one or more storage systems comprising any of a variety of types of storage including network-attached storage, storage area networks, direct-attached storage, cloud storage, and distributed direct-attached storage, as well as combinations of these and other storage types, including software-defined storage.

Additionally or alternatively, in one or more embodiments, data related to treatment sessions and/or users can be read, processed and/or otherwise used (e.g., in connection with one or more input-output devices as detailed below) by one or more humans and/or one or more computing devices, for example, to make one or more adjustments (e.g., automatically by program and/or by human-initiated action) to the treatment device and/or one or more functions thereof.

Also associated with the treatment device, in at least one embodiment, can be one or more input-output devices, which can include, by way merely of example, keyboards, displays or other types of input-output devices in any combination. Such input-output devices can be used to support one or more user interfaces (UIs) to the treatment device, as well as to support communication between the treatment device and other related systems and devices not explicitly illustrated in FIGS. 1A-1C, FIG. 2, FIG. 3, FIG. 4 or FIG. 5.

It is to be understood that the particular sets of elements shown in FIGS. 1A-1C, FIG. 2, FIG. 3, FIG. 4 and FIG. 5 are depicted by way of illustrative example only, and in one or more other embodiments, additional or alternative elements may be used.

Accordingly, at least one embodiment includes a cable-based (e.g., a single coaxial cable-based) and radio frequency-based therapeutic device for the treatment of neurological conditions. As further detailed herein, such an embodiment includes generating and/or implementing a treatment device which includes at least one small PIN diode (e.g., a sub-millimeter surface mount component, etc.). As used herein, a PIN diode refers to a diode with an undoped intrinsic semiconductor region between a p-type semiconductor region and an n-type semiconductor region. In at least one embodiment, the PIN diode enables control of the treatment device, including selective activation of any or all of one or more antennas, using at least one cable (e.g., a single coaxial cable).

Additionally, in one or more embodiments, implementing the PIN diode enables RF-based treatment patterns focused on the entire brain or one or more selective portions thereof, thereby facilitating versatility of the treatment device that can provide patient-to-patient customized treatments. As further described herein, at least one embodiment provides, relative to conventional devices and treatments, a reduced number of points of device wear-out and/or failure.

The treatment device of one or more embodiments can also be implemented in a variety of ways. For example, a radio-frequency generator-controller unit (RF generator-controller unit), as further detailed herein as a component of the treatment device, can be separately affixed to a user (for example, strapped on the user's arm via a band, strap, etc., as depicted, e.g., in FIG. 1A) and/or embedded inside of the treatment device itself (for example, as an all-in-one wearable therapeutic, as depicted, e.g., in FIG. 1C). Additionally or alternatively, one or more embodiments include precluding and/or overcoming limiting factors of conventional devices and/or treatments such as, for example, antenna type and operational frequency dependency, as such an embodiment works across all antenna types and all reasonable-to-use frequencies. By way of example, in one or more embodiments, reasonable-to-use frequencies can include radio frequencies in the extremely low frequency (ELF) band, super low frequency (SLF) band, ultra-low frequency (ULF) band, very low frequency (VLF) band, low frequency (LF) band, medium frequency (MF) band, high frequency (HF) band, very high frequency (VHF) band, ultra-high frequency (UHF) band, and/or super high frequency (SHF) band. Accordingly, at least one embodiment includes generating and/or implementing a treatment device that is antenna-type agnostic, frequency agnostic, treatment-distance agnostic, and signal-strength agnostic. Such a treatment device, as noted, may be a wearable (e.g., a waterproof and/or washable wearable) and/or may be embedded in a proximate device or structure (e.g., bedframes, pillows, head supports for wheelchairs, etc.).

By way of greater specificity, FIG. 1A depicts an example RF generator-controller unit 102 affixed to a user's arm, and a cable 109 is also depicted to show how the RF generator-controller unit 102 might attach to a separate (e.g., head-worn) therapeutic portion, such as depicted in FIG. 1B via therapeutic component 110. As further detailed herein, in one or more embodiments, the RF generator-controller unit 102 can also be worn around a user's neck, incorporated in a waist band, embedded in and/or carried by clothing or an accessory (e.g., a handbag), and/or carried, affixed, or transported in other user-convenient ways.

It is to be appreciated (and as further detailed herein) that the embodiments depicted in FIG. 1 serve as merely example implementations. For example, in one or more alternate embodiments, RF generator-controller unit 102 can be implemented and/or encompassed in one or more fixed and non-wearable locations (e.g., in one or more articles of clothing, in one or more items of furniture, in one or more structural components, etc.).

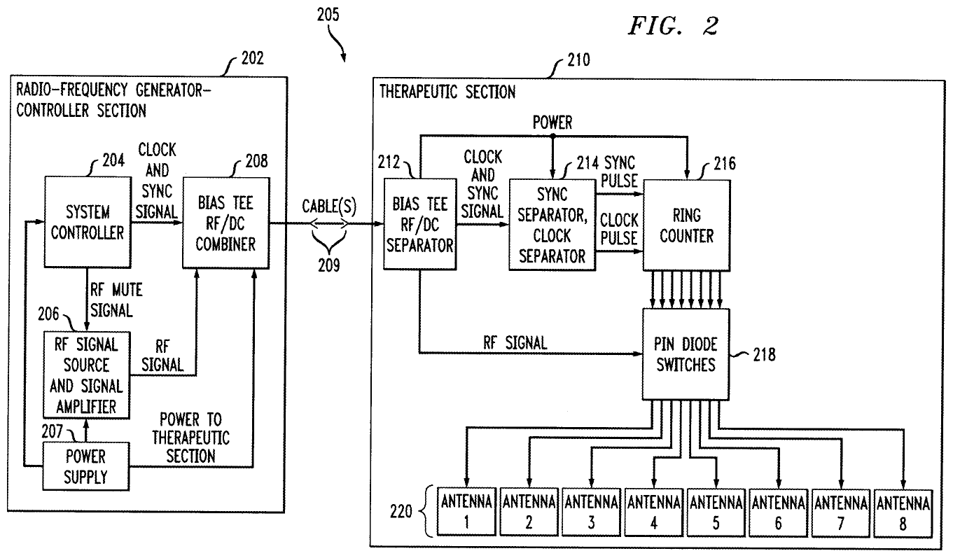

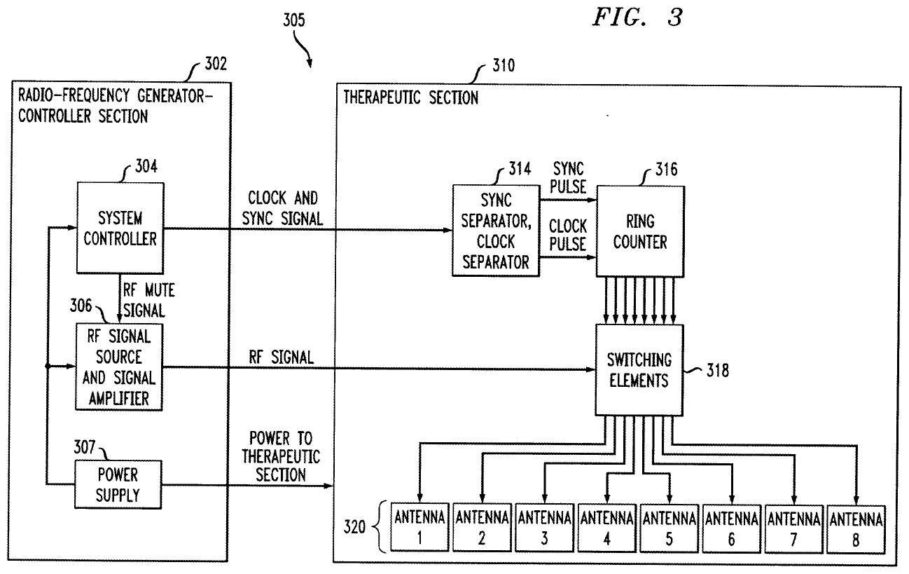

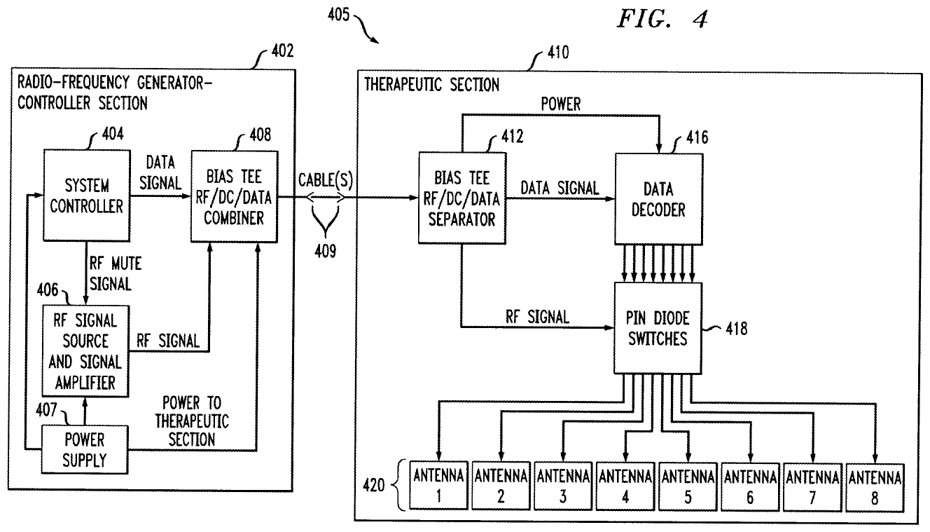

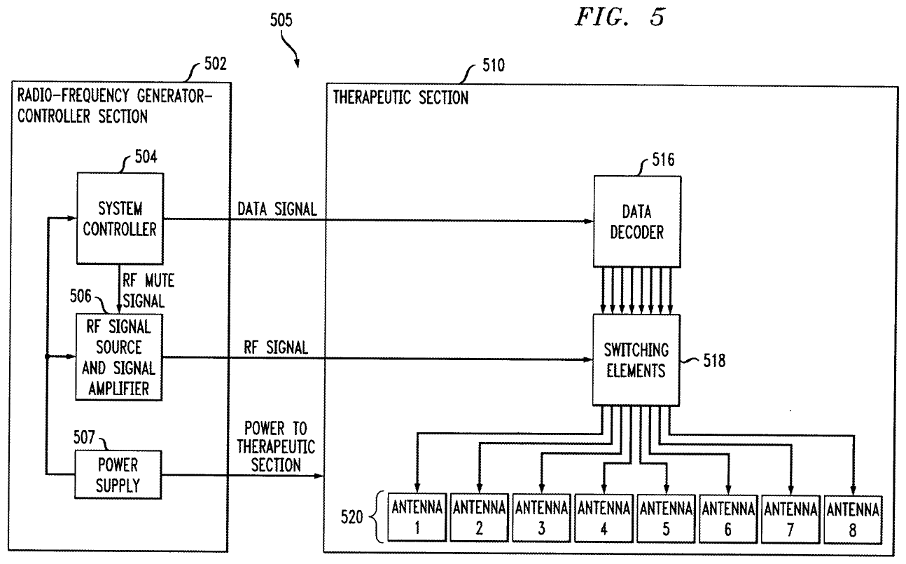

As also illustrated, FIG. 1C depicts an all-in-one treatment device 105. In this particular embodiment, illustrated merely by way of example and not limitation, the treatment device 105 is wearable and takes the form of a baseball cap or one or more components thereof. It is to be appreciated, though, that one or more additional and/or alternative designs and/or implementations can be envisioned and embodied. All RF generator-controller section (also referred to herein as RF generator-controller unit) circuitry (e.g., element 102 in FIG. 1A, element 202 in FIG. 2, element 302 in FIG. 3, element 402 in FIG. 4, and element 502 in FIG. 5) can be incorporated, for instance, via at least one flexible printed circuit board (PCB), and the at least one flexible PCB can be embedded, for example, within or affixed to the cap's strap or bill/visor. In one or more embodiments, at least one battery power supply (such as, e.g., a rechargeable lithium-polymer (LiPo) battery) can also be incorporated with the cap's strap and/or bill/visor. The therapeutic section (e.g., element 110 in FIG. 1B, element 210 in FIG. 2, element 310 in FIG. 3, element 410 in FIG. 4, and element 510 in FIG. 5) of the treatment device can be incorporated, for example, within the cap's head-covering material. With proper selection of materials (e.g., water-resistant and/or waterproof materials) and proper embedding of the electronics, the treatment device 105 can be cleanable and/or washable. In one or more embodiments, such an example treatment device 105 can also support wireless charging for further user convenience.

As also detailed herein, one or more embodiments include implementing one or more PIN diodes as part of a treatment device. However, it is to be appreciated by one skilled in the art that one or more RF-switching alternatives to PIN diodes can be utilized by one or more embodiments as switching elements. In one or more embodiments, RF-switching alternatives can include, but are not limited to, field-effect transistors (FETs), metal-oxide-semiconductor field-effect transistors (MOSFETs), bipolar transistors, complementary metal-oxide-semiconductors (CMOS), PIN-diode/FET/MOSFET/bipolar transistors/CMOS combinations, and electromechanical switches. As used herein, “switching elements” generally refer to devices that allow radio frequency energy to be toggled on and off and/or passed or not passed.

At least one embodiment can also include incorporating at least one diagnostic component, sensor component, and/or system-measurement component into the treatment device. For example, such a diagnostic component can include at least one tool or sensor for measuring various biological signals from a user (e.g., temperature, pulse rate, blood oxygen, or other biological characteristics). In another example, at least one sensor can be incorporated that verifies that the treatment device is actually being worn while activated. In a further example, at least one sensor can be incorporated that measures the RF field strength associated with one or more RF delivery components of the treatment device.

Additionally, in one or more embodiments, because at least a portion of the therapeutic sections (e.g., antennas and PIN diodes) of the treatment devices are connected to an RF generator and/or RF controller section by at least one cable (e.g., a single coaxial cable), a therapeutic section can be, for example, unplugged and/or removable (from the RF generator and/or RF controller section) and cleanable and/or washable (e.g., by encasing the therapeutic section in, for example, a silicone sheath).

FIG. 2 is a block diagram illustrating an example circuit of a treatment device in an example embodiment. By way of illustration, such an example circuit includes using PIN diodes as one or more switching elements. Specifically, as depicted in FIG. 2, an example treatment device 205 can include an RF generator-controller section 202, which can include a system controller 204, an RF signal source and signal amplifier 206 (e.g., a signal source and an amplifier of the generated signal(s)), power supply 207 (e.g., a battery and/or hard-wired power source), and a bias tee RF/direct current (RF/DC) combiner 208. As used herein, a bias tee generally refers to a technology element that is used to allow and/or facilitate RF and DC (e.g., radio frequency energy, power, system signals, etc.) to travel in a cable. Also, FIG. 2 depicts a therapeutic section 210, connected to RF generator-controller section 202 via one or more cables 209 (e.g., with one or more corresponding connectors), and which includes a bias tee RF/DC separator 212, a sync separator and clock separator 214, a ring counter 216, one or more PIN diode switches 218 and a corresponding number of antennas 220. It is to be appreciated that in one or more embodiments, the number of antennas (e.g., component 220 in FIG. 2) has no predetermined minimum or maximum value. In such an embodiment, as the selected frequency increases, antenna size decreases (as the wavelengths are smaller), and even more antennas can fit on the therapeutic section (e.g., element 210 in FIG. 2).

Also, in one or more embodiments and as further detailed in connection with FIG. 2, FIG. 3, FIG. 4 and FIG. 5, DC power is generated and/or provided by a power supply (e.g., element 207, 307, 407 and/or 507) and is routed through one or more bias tee components (e.g., elements 208 and 212 in FIG. 2 and/or elements 408 and 412 in FIG. 4) or directly via wires (e.g., as depicted in the example FIG. 3 and FIG. 5 embodiments).

As detailed herein and depicted in FIG. 2, one or more embodiments can include requiring only one coaxial cable to connect the RF generator-controller section 202 and the therapeutic section 210, as the signals, DC power, and RF travel over the single cable (e.g., a cable as represented by element 209 in FIG. 2). In an example of such an embodiment, cable connectors can include a male component and a female component, and as such, may be referred to herein as a mating coaxial-connector set. Alternatively, in one or more embodiments, no mating coaxial-connector set is needed, as such an embodiment can include using an unbroken length of cable or wire. Embodiments that include utilizing a connector set, however, enable and/or facilitate a means to wash and/or clean the therapeutic component, provide an intentional break-away connector in case the user tugs on the cable, provide easier sub-system repairs and/or upgrades, etc.

As further detailed herein, RF generator-controller section 202 can serve multiple purposes, such as, for example, providing necessary control signals, antenna switching signals, and RF power for the therapeutic section 210 of the treatment device 205. In one or more embodiments, the system controller 204 orchestrates various functions and operations of the treatment device 205. Also, the RF signal source and signal amplifier 206 that provides a source and signal strength of the chosen RF frequency and chosen signal strength for therapeutic action, and power supply 207 provides power to one or more of the systems within RF generator-controller section 202 and therapeutic section 210.

As also depicted in FIG. 2, a portion of the DC power is combined in the bias tee RF/DC combiner 208 with the RF signal(s), and the two travel through the cable(s) 209 to bias tee RF/DC separator 212, wherein the inputs are separated. The DC power then comes out of bias tee RF/DC separator 212 and is distributed to multiple components (e.g., integrated circuits (ICs), resistors, capacitors, inductors, transformers, diodes, etc.) within therapeutic section 210.

The bias tee RF/DC combiner 208, in the RF generator-controller section 202, couples RF power with the DC, clock and sync signals (derived and/or obtained from system controller 204), which allows the RF power and the DC, clock and sync signals to travel in the same cable(s) 209 (e.g., with corresponding connectors) to the therapeutic section 210 of the treatment device 205. In one or more embodiments, the output of RF signals includes a short pause (e.g., one or more milliseconds pause) when the PIN diodes are switched. In such an embodiment, it is important to turn off the RF power when the PIN diodes switch, and a mute function, directed by system controller 204, momentarily turns off RF power when PIN diodes are about to switch, such that the PIN diodes are not under load during the switching actions. The mute function also allows selective deactivation of specific antennas, if required. For instance, in an eight-antenna system, if a user requires that only antennas 4, 5, and 6 are used in a treatment, and not antennas 1, 2, 3, 7, and 8, then system controller 204 would turn off power to antennas 1, 2, 3, 7, and 8 through the mute function. Similarly, if a user requires that all antennas are to be turned off, system controller 204 would direct the mute function to not allow RF to flow into any of the antennas.

Additionally, the bias tee RF/DC separator 212, in the therapeutic section 210, separates the received RF and power signals from the received DC, clock and sync signals. The bias tee RF/DC separator 212 sends power to sync separator and clock separator 214 and to ring counter 216, and sends one or more RF signals to PIN diode switches 218. Also, sync separator and clock separator 214 separates the DC, clock and sync signals, and the separated clock and sync signals are fed to ring counter 216.

Referring again to FIG. 2, ring counter 216 is incremented by received clock signals, and the ring counter 216 activates the appropriate PIN diode switch(es) to make the appropriate antenna selection(s) and activation(s). In an example embodiment, the system controller 204 is programmed to deliver RF emissions in a prescribed sequence to the antennas 220. The ring counter 216 interprets the incoming pulses and/or signals prescribed by the system controller 204, and causes the appropriate PIN diode switches 218 to be forward biased; this conductive state allows RF emissions to flow to the mated antennas 220. It should be noted that the system controller 204 can be programmed to enable delivery of RF emissions to a particular selection of antennas or to all of the antennas 220.

Additionally, in one or more embodiments, system signals can be adjusted to change the timing and/or selection of the desired RF states of the various antennas. Also, in at least one embodiment, the sync signal(s) can reset the ring counter 216 back to an initial antenna state.

As detailed herein, and as in part depicted in FIG. 2, the cable(s) 209 (e.g., with corresponding connectors) between RF generator-controller section 202 and therapeutic section 210 can be of one continuous length, or, a mating coaxial-connector set can be applied to detach and reattach RF generator-controller section 202 and therapeutic section 210.

FIG. 3 is a block diagram illustrating an example circuit of a treatment device in an example embodiment. By way of illustration, FIG. 3 depicts an example treatment device 305 which is similar to treatment device 205 depicted in FIG. 2, but without the inclusion and/or use of a bias tee RF/DC combiner or a bias tee RF/DC separator. It is to be appreciated that the elements depicted in FIG. 3 that are also depicted in FIG. 2 can be described similarly as above in connection with FIG. 2. In an example embodiment such as depicted in FIG. 3, RF emissions can be switched on and off, toggled from one antenna to another (e.g., among antennas 320), based, for example, on instructions from the system controller 304. Also, in an example embodiment such as depicted in FIG. 3, wires associated with system controller 304, RF signal source and signal amplifier 306 (e.g., a signal source and an amplifier of the generated signal(s)), and power supply 307 can have narrow diameters and be bundled (e.g., such as with heat-shrink tubing) to have an outward appearance to the user as being one single cable. For example, such wires (as represented by arrows in FIG. 3) can include a wire that provides clock and sync signals from system controller 304 to sync separator, clock separator 314 (which separates the sync signal(s) and clock signal(s) and sends them to antennas 320 via ring counter 316 and switching elements 318), a wire that provides RF signals from RF signal source and signal amplifier 306 to switching elements 318, and a wire that provides power from power supply 307 to one or more elements of therapeutic section 310 and/or one or more elements of RF generator-controller section 302.

FIG. 4 is a block diagram illustrating another example circuit of a treatment device in an example embodiment. FIG. 4 depicts an example embodiment that does not require sync and clock pulses or a ring counter, such as depicted in the example FIG. 2 embodiment, facilitating enhanced bi-directional data flow (e.g., if sensors are built into the therapeutic device). As further detailed below, in the example FIG. 4 embodiment, the coaxial cable is implemented as a combined data line, RF line, and power line. More specifically, in the FIG. 4 embodiment, the system controller 404 provides the data necessary to control the PIN diode switches 418.

Accordingly, and by way of further illustration, the example circuit depicted in FIG. 4 includes using PIN diode switches 418 as one or more switching elements. Specifically, in such an embodiment, treatment device 405 can include RF generator-controller section 402, which can include system controller 404, RF signal source and signal amplifier 406 (e.g., a signal source and an amplifier of the generated signal(s)), power supply 407, and bias tee RF/DC/DATA combiner 408. Also, FIG. 4 depicts therapeutic section 410, connected to RF generator-controller section 402 via a mating coaxial-connector set in connection with cable(s) 409, and which includes bias tee RF/DC/DATA separator 412, data decoder 416, one or more PIN diode switches 418, and a corresponding number of antennas 420. For example, in one or more embodiments, a particular mating coaxial-connector set can depend on the diameter of the cable, the use of the cable, and/or the frequency of the RF.

It is to be appreciated, also, that in one or more alternative embodiments, mating coaxial-connector set can be replaced via the use of an uninterrupted length of cable.

As further detailed herein, RF generator-controller section 402 can serve multiple purposes, such as, for example, providing necessary control signals, antenna switching signals, and/or RF power for the therapeutic section 410 of the treatment device 405. Additionally, the system controller 404 orchestrates various functions and operations of the treatment device 405. Also, the RF signal source and signal amplifier 406 that provides a source and signal strength of the chosen RF frequency and chosen signal strength for therapeutic action, and power supply 407 provides power to one or more of the systems within RF generator-controller section 402 and therapeutic section 410.

The bias tee RF/DC/DATA combiner 408, in the RF generator-controller section 402, couples RF power with the DC and data signals (derived and/or obtained from system controller 404), which allows the RF power, DC, and data signals to travel in the same cable(s) 409 (e.g., with corresponding connectors) to the therapeutic section 410 of the treatment device 405. Additionally, the bias tee RF/DC/DATA separator 412, in the therapeutic section 410, separates the RF and power signals from the received DC and data signals. The bias tee RF/DC/DATA separator 412 sends power to data decoder 416, and sends one or more RF signals to PIN diode switches 418. Data decoder 416 activates the appropriate PIN diode switch(es) 418 to make the appropriate antenna selection(s) and activation(s).

As noted, in at least one embodiment, the system controller 404 is programmed to deliver RF emissions in a prescribed sequence to the antennas 420. The data decoder 416 interprets the incoming signals prescribed by the system controller 404 and causes the appropriate PIN diode switches 418 to be forward biased; this conductive state allows RF emissions to flow to the mated antennas 420. It should be noted that the system controller 404 can be programmed to enable delivery of RF emissions to a particular selection of antennas or to all of the antennas 420. Additionally, in one or more embodiments, system signals can be adjusted to change the timing and/or selection of the desired RF states of the various antennas.

Also, the cable(s) 409 between RF generator-controller section 402 and therapeutic section 410 can be of one continuous length, or a mating coaxial-connector set can be applied (such as depicted in FIG. 4) to detach and reattach RF generator-controller section 402 and therapeutic section 410.

FIG. 5 is a block diagram illustrating an example circuit of a treatment device in an example embodiment. By way of illustration, FIG. 5 depicts an example treatment device 505 which is similar to treatment device 405 depicted in FIG. 4, but without the inclusion and/or use of a bias tee RF/DC/DATA combiner or a bias tee RF/DC/DATA separator. It is to be appreciated that the elements depicted in FIG. 5 that are also depicted in FIG. 4 can be described similarly as above in connection with FIG. 4. In an example embodiment such as depicted in FIG. 5, RF emissions can be switched on and off, toggled from one antenna to another (e.g., among antennas 520), based, for example, on instructions from the system controller 504. Also, in an example embodiment such as depicted in FIG. 5, wires associated with system controller 504, RF signal source and signal amplifier 506 (e.g., a signal source and an amplifier of the generated signal(s)), and power supply 507 can have narrow diameters and be bundled (e.g., such as with heat-shrink tubing) to have an outward appearance to the user as being one single cable. For example, such wires (as represented by arrows in FIG. 5) can include a wire that provides data signals from system controller 504 to data decoder 516, a wire that provides RF signals from RF signal source and signal amplifier 506 to switching elements 518, and a wire that provides power from power supply 507 to one or more elements of therapeutic section 510 and/or one or more elements of RF generator-controller section 502.

As detailed herein, one or more embodiments include generating and/or implementing an RF-based therapeutic device. In an example embodiment, such a device includes one or more antenna elements sized and configured for one or more operational frequencies, at least one switching element coupled with at least a portion of the one or more antenna elements, at least one RF generator-controller unit, and at least one cable, wherein the at least one cable facilitates transmission of one or more of RF emissions, one or more antenna control voltages, and one or more system-control signals between the at least one RF generator-controller unit and the at least one switching element in connection with at least a portion of the one or more antenna elements.

In one or more embodiments, such configuration can include, for example, placement and spacing of one or more antenna elements (also referred to herein simply as antennas) such that the one or more antenna elements are optimized for therapeutic effect. Additionally or alternatively, such configuration can include, for example, orienting one or more antenna elements for increased and/or optimum therapeutic effect, increased and/or optimum power-to-therapy efficiency (e.g., to maximize battery life), and/or for reducing and/or minimizing non-therapeutic RF entering the immediate environment.

In at least one embodiment, the size of the one or more antenna elements can vary based at least in part on one or more design considerations, and can be approximately inversely proportional to frequency. In at least one embodiment, the size of the cable(s) used can vary depending, for example, on one or more design considerations, impedance of a corresponding circuit, etc.

In at least one embodiment, the at least one cable includes at least one coaxial cable (e.g., a single multi-functional coaxial cable). Alternatively, in one or more embodiments, the at least one cable can include at least one triaxial cable (e.g., a single multi-functional triaxial cable). Further still, in at least one embodiment, the at least one cable can include multiple cables that are each dedicated to a particular purpose (e.g., a cable dedicated to RF, a cable dedicated to data, a cable dedicated to power, etc.) and/or two or more parallel elements of a given cable.

Accordingly, and as further detailed herein, one or more embodiments includes reducing and/or eliminating point-to-point architecture (e.g., one wire from the transmitter to each antenna). Such an embodiment can include distributing the logic and control into multiple parts (e.g., two parts) such that the corresponding device includes digital-logic components that communicate to the controller (e.g., a wearable controller) in addition to the antennas.

Also, in one or more embodiments, the at least one switching element includes at least one solid-state switch and/or at least one electromechanical switch. The at least one solid-state switch can include, for example, at least one PIN diode, at least one FET, at least one MOSFET, at least one bipolar transistor, at least one CMOS, and/or at least one combination thereof.

In at least one embodiment, at least a portion of the one or more antenna elements and at least a portion of the at least one switching element are embedded in one or more physical components and/or device housings. For example, in one or more embodiments, at least a portion of the one or more antenna elements, at least a portion of the at least one switching element, at least a portion of the at least one RF generator-controller unit, and at least a portion of the at least one cable are comprised in a single physical device. Additionally or alternatively, at least a portion of the one or more antenna elements and at least a portion of the at least one switching element are comprised in at least a first physical device, and at least a portion of the at least one RF generator-controller unit is comprised in at least a second physical device, and wherein the at least a first physical device and the at least a second physical device are connected via the at least one cable. In such embodiments, the one or more physical devices can include, by way merely of example, at least one head covering, at least one piece of furniture (e.g., the headboard of a bed, the mattress of a bed, the head-support structure of a chair, the head-support structure of a wheelchair), at least one pillow, and/or at least one blanket.

Additionally or alternatively, in one or more embodiments, an RF-based therapeutic device can include and/or be linked to a separate and/or stand-alone data recording unit. Data recording units can include, for example, a mobile device running a software application, a cloud-based server (e.g., implemented in connection with one or more artificial intelligence techniques), etc.

Also, in one or more embodiments, at least a portion of the one or more antenna elements are waterproof and/or at least a portion of the at least one switching element are waterproof Additionally or alternatively, at least a portion of the at least one switching element at each of the one or more antenna elements can be configured in a waterproof manner. For example, such waterproofing can include sealing the element(s) in question from water using, for instance, at least one flexible membrane, etc. Further, in at least one embodiment, the device is configured for treatment of one or more neurological conditions.

Additionally, in one or more embodiments, such an RF-based therapeutic device can include one or more sensors. For example, the therapeutic portion of the device can be capable of sending data back to the controller portion of the device (e.g., using universal asynchronous receiver-transmitters). In such an embodiment, the one or more sensors can measure one or more biological characteristics of the user. For example, the one or more sensors can include one or more proximity detectors (which can help determine whether or not the user is actually wearing the device when the RF-treatment is activated) and/or one or more sensors that measure RF field strength.

As also detailed herein, one or more embodiments include generating and/or implementing an RF-based therapeutic device which includes one or more antenna elements sized and configured for one or more operational frequencies, at least one RF generator-controller unit, at least one switching element (e.g., at least one solid-state switch or at least one electromechanical switch) coupled with at least a portion of the one or more antenna elements, and a distributed functional architecture, wherein the distributed functional architecture facilitates transmission of one or more of RF emissions, one or more antenna control voltages, and one or more system-control signals between the at least one RF generator-controller unit and the at least one switching element in connection with at least a portion of the one or more antenna elements. In such an embodiment, the one or more antenna elements, the at least one RF generator-controller unit, the at least one switching element, and the distributed functional architecture are incorporated on one or more PCBs. Additionally, in such an embodiment, the one or more PCBs can include at least one connection with at least one power source (e.g., one or more batteries) and one or more cables associated with the one or more antenna elements (e.g., one or more small-diameter coaxial cables connecting to the one or more antenna elements). Further, in at least one embodiment, such an RF-based therapeutic device can include one or more sensors (such as detailed herein, for example).

In one or more embodiments, a distributed functional architecture can include, for example, a power supply, a system controller, an RF signal source, an RF signal amplifier, an antenna-switch system, and one or more antennas that are appropriately packaged, configured, and distributed within the RF-based therapeutic device.

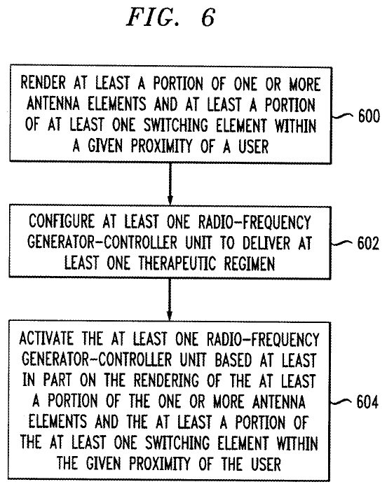

FIG. 6 is a flow diagram of a process for implementing a treatment device in an illustrative embodiment of the invention. In this embodiment, the process includes steps 600 through 604. These steps are assumed to be performed by at least portions of at least one RF-based therapeutic device (e.g., treatment device 105, 205, 305, 405 and/or 505).

Step 600 includes rendering at least a portion of one or more antenna elements and at least a portion of at least one switching element within a given proximity of a user. In one or more embodiments, the given proximity can include a range of a fraction of an inch (e.g., for a cap worn on a user's head) to multiple feet (e.g., for a device incorporated in, for example, a bed frame).

Step 602 includes configuring at least one RF generator-controller unit to deliver at least one therapeutic regimen (e.g., at least one therapeutic regimen associated with the user). In one or more embodiments, the at least one therapeutic regimen includes multiple variables comprising two or more of RF frequency, RF signal strength, RF duration, RF pulsing, one or more antenna selections, and one or more antenna treatment patterns. Additionally or alternatively, in at least one embodiment, the at least one therapeutic regimen can include multiple data-recording process variables comprising two or more of one or more RF parameters, ambient temperature, user temperature, user-related schedule, one or more user-related usage patterns (e.g., treatment session adherence), and one or more system diagnostic parameters.

Step 604 includes activating the at least one RF generator-controller unit based at least in part on the rendering of the at least a portion of the one or more antenna elements and the at least a portion of the at least one switching element within the given proximity of the user. In one or more embodiments, activating the at least one RF generator-controller unit is carried out by at least one of a local therapy-control center and a remote therapy-control center. In such an embodiment a local therapy-control center and/or a remote therapy-control center can include doctors' offices, regional dementia-treatment centers (e.g., a hospital and/or large-scale medical institution), research centers, etc.

Additionally or alternatively, in at least one embodiment, activating the at least one RF generator-controller unit can include enabling the at least one RF generator-controller unit to conduct one or more of at least one user-specific therapeutic regimen, therapeutic regimen-related data processing, system update processing, one or more data transfers, one or more data uploads, and one or more data downloads.

In one or more embodiments, the techniques depicted in FIG. 6 can also include transmitting data, via the RF generator-controller unit in connection with the at least one therapeutic regimen, to one or more data processing systems. In such an embodiment, the one or more data processing systems can be, for example, manually operated and/or automatically operated (e.g., via one or more machine learning and/or artificial intelligence techniques). Also, in at least one embodiment, the one or more data processing systems can include, for example, at least one data center and/or at least one artificial intelligence-based integrated platform configured to expand and/or enhance data transmission to and/or from one or more devices (e.g., devices associated with various treatments for the user).

Other techniques can be used in association with one or more embodiments of the invention. Accordingly, the particular processing operations and other network functionality described in conjunction with FIG. 6 are presented by way of illustrative example only, and should not be construed as limiting the scope of the invention in any way. For example, the ordering of the process steps may be varied in one or more other embodiments of the invention, or certain steps may be performed concurrently with one another rather than serially. Also, the process steps or subsets thereof may be repeated periodically in conjunction with respective distinct instances of treatment devices with respect to different users.

The above-described example embodiments of the invention provide significant advantages relative to conventional approaches. For example, one or more embodiments of the invention can include reducing and/or eliminating point-to-point architecture. Additionally, one or more embodiments include distributing logic and control into multiple components such that the device associated therewith includes digital-logic components that communicate to the controller (e.g., a wearable controller) in addition to the antennas.

It is to be appreciated that the foregoing advantages are illustrative of advantages provided in certain embodiments, and need not be present in other embodiments.

Further, in accordance with one or more embodiments, processing devices, and other network components can communicate with one another using a variety of different communication protocols and associated communication media, and overseen by human and/or computer-driven resources.

In one or more embodiments of the invention, portions of a network as disclosed herein can illustratively include cloud infrastructure. The cloud infrastructure, in at least one such embodiment of the invention, can include a plurality of containers implemented using container host devices, and/or can include container-based virtualization infrastructure configured to implement Docker containers or other types of Linux containers.

The cloud infrastructure can additionally or alternatively include other types of virtualization infrastructure such as virtual machines implemented using a hypervisor. Additionally, the underlying physical machines include one or more distributed processing platforms that include one or more storage systems.

It should again be emphasized that the embodiments described herein are presented for purposes of illustration only. Many variations may be made in the particular arrangements shown. Moreover, the assumptions made herein in the context of describing one or more illustrative embodiments should not be construed as limitations or requirements of the invention, and need not apply in one or more other embodiments. Numerous other alternative embodiments within the scope of the appended claims will be readily apparent to those skilled in the art.

What is claimed is:

1. A radio-frequency-based (RF-based) therapeutic device, comprising:
one or more antenna elements sized and configured for one or more operational frequencies;
at least one RF generator-controller unit;
at least one switching element coupled with at least a portion of the one or more antenna elements; and
at least one cable, wherein the at least one cable facilitates transmission of one or more of RF emissions, one or more antenna control voltages, and one or more system-control signals between the at least one RF generator-controller unit and the at least one switching element in connection with at least a portion of the one or more antenna elements.

2. The RF-based therapeutic device of claim 1, wherein the at least one cable comprises a single coaxial cable.

3. The RF-based therapeutic device of claim 1, wherein the at least one switching element comprises at least one solid-state switch.

4. The RF-based therapeutic device of claim 3, wherein the at least one solid-state switch comprises one or more of at least one PIN diode, at least one field-effect transistor (FET), at least one metal-oxide-semiconductor field-effect transistor (MOSFET), at least one bipolar transistor, at least one complementary metal-oxide-semiconductor (CMOS), and at least one combination thereof.

5. The RF-based therapeutic device of claim 1, wherein the at least one switching element comprises at least one electromechanical switch.

6. The RF-based therapeutic device of claim 1, wherein at least a portion of the one or more antenna elements, at least a portion of the at least one switching element, at least a portion of the at least one RF generator-controller unit, and at least a portion of the at least one cable are comprised in a single physical device.

7. The RF-based therapeutic device of claim 1, wherein at least a portion of the one or more antenna elements and at least a portion of the at least one switching element are comprised in at least a first physical device, and at least a portion of the at least one RF generator-controller unit is comprised in at least a second physical device, and wherein the at least a first physical device and the at least a second physical device are connected via the at least one cable.

8. The RF-based therapeutic device of claim 1, wherein the device is configured for treatment of one or more neurological conditions.

9. The RF-based therapeutic device of claim 1, further comprising:
one or more sensors.

10. The RF-based therapeutic device of claim 9, wherein the one or more sensors measure at least one of one or more biological characteristics of a user, one or more proximity-related variables, and RF field strength.

11. An RF-based therapeutic device, comprising:
one or more antenna elements sized and configured for one or more operational frequencies;
at least one RF generator-controller unit;
at least one switching element coupled with at least a portion of the one or more antenna elements; and
a distributed functional architecture, wherein the distributed functional architecture facilitates transmission of one or more of RF emissions, one or more antenna control voltages, and one or more system-control signals between the at least one RF generator-controller unit and the at least one switching element in connection with at least a portion of the one or more antenna elements.

12. The RF-based therapeutic device of claim 11, wherein the one or more antenna elements, the at least one RF generator-controller unit, the at least one switching element, and the distributed functional architecture are incorporated on one or more printed circuit boards.

13. The RF-based therapeutic device of claim 12, wherein the one or more printed circuit boards comprises at least one connection with at least one power source and one or more cables associated with the one or more antenna elements.

14. The RF-based therapeutic device of claim 11, wherein the at least one switching element comprises one of at least one solid-state switch and at least one electromechanical switch.

15. The RF-based therapeutic device of claim 11, further comprising:
one or more sensors.

16. A method comprising:
rendering at least a portion of one or more antenna elements and at least a portion of at least one switching element within a given proximity of a user;
configuring at least one RF generator-controller unit to deliver at least one therapeutic regimen; and
activating the at least one RF generator-controller unit based at least in part on the rendering of the at least a portion of the one or more antenna elements and the at least a portion of the at least one switching element within the given proximity of the user;
wherein the method is carried out by at least one RF-based therapeutic device.

17. The method of claim 16, wherein the at least one therapeutic regimen comprises multiple variables comprising two or more of RF frequency, RF signal strength, RF duration, RF pulsing, one or more antenna selections, and one or more antenna treatment patterns.

18. The method of claim 16, wherein the at least one therapeutic regimen comprises multiple data-recording process variables comprising two or more of one or more RF parameters, ambient temperature, user temperature, user-related schedule, one or more user-related usage patterns, and one or more system diagnostic parameters.

19. The method of claim 16, wherein activating the at least one RF generator-controller unit comprises enabling the at least one RF generator-controller unit to conduct one or more of at least one user-specific therapeutic regimen, therapeutic regimen-related data processing, system update processing, one or more data transfers, one or more data uploads, and one or more data downloads.

20. The method of claim 16, further comprising:
transmitting data, via the RF generator-controller unit in connection with the at least one therapeutic regimen, to one or more data processing systems.
